(19) United States
(12) Patent Application Publication
(10) Pub. No.: US 2005/0055165 A1
(43) Pub. Date: Mar. 10, 2005

(54) Calculating a potential of mean force (PMF) score of a protein-ligand complex

(76) Inventor: George D. Purvis III, Beaverton, OR (US)

Correspondence Address: BAKER BOTTS L.L.P., 2001 ROSS AVENUE, SUITE 600, DALLAS, TX, 75201-2980, US

(21) Appl. No.: 10/655,870

(22) Filed: Sep. 5, 2003

(51) Int. Cl. G01N 33/48; G01N 33/50; G01N 31/00

(52) U.S. Cl. 702/19

(57) ABSTRACT

In one embodiment, a system for calculating a potential of mean force (PMF) score of a protein-ligand complex includes a repulsion-term module that accesses one or more parameters useable to calculate a repulsion term useable to calculate a PMF of a protein-ligand atom pair in the protein-ligand complex. The one or more parameters correspond to an atom-pair type of the protein-ligand atom pair. The repulsion-term module uses the one or more accessed parameters to calculate the repulsion term useable to calculate the PMF of the protein-ligand atom pair. Th repulsion-term module communicates the calculated repulsion term for calculation of the PMF score of the protein-ligand complex.

### TECHNICAL FIELD OF THE INVENTION

This invention relates in general to chemical interactions and in particular to calculating a PMF score of a protein-ligand complex.

### BACKGROUND OF THE INVENTION

A drug that may be used to treat or cure illness may include one or more ligands that may bind to one or more proteins to inhibit or otherwise modify the function of the proteins. The binding affinity between a ligand and a protein may determine, at least in part, the ability of the ligand to modify the function of the protein. As an example, a ligand that has greater binding affinity to a protein may be more effective at modifying the function of the protein than a ligand that has less binding affinity to the protein. As a result, a ligand that has greater binding affinity to a protein associated with an illness may be a better candidate for a drug that may be used to treat or cure the illness than a ligand that has less binding affinity to the protein.

To identify a ligand that may have greater binding affinity to a protein associated with the illness, potential mean force (PMF) scores of multiple protein-ligand complexes that each include the protein and one of multiple ligands may be calculated and compared with each other. A PMF score of a protein-ligand complex may indicate the binding affinity between the protein and ligand. A ligand in a protein-ligand complex that has a lower (more negative) PMF score may be a better candidate for a drug that may be used to treat the illness than a ligand in a protein-ligand complex that has a higher PMF score.

### SUMMARY OF THE INVENTION

Particular embodiments of the present invention may reduce or eliminate disadvantages and problems associated with calculating a PMF score of a protein-ligand complex.

In one embodiment of the present invention, a system for managing demand influence includes a repulsion-term module that accesses one or more parameters useable to calculate a repulsion term useable to calculate a PMF of a protein-ligand atom pair in the protein-ligand complex. The one or more parameters correspond to an atom-pair type of the protein-ligand atom pair. The repulsion-term module uses the one or more accessed parameters to calculate the repulsion term useable to calculate the PMF of the protein-ligand atom pair. Th repulsion-term module communicates the calculated repulsion term for calculation of the PMF score of the protein-ligand complex.

Particular embodiments of the present invention may provide one or more technical advantages. Particular embodiments may be used to more accurately predict a structure of a protein-ligand complex. Particular embodiments may be used to more accurately calculate a binding affinity between a protein and a ligand and the positions of the atoms in the protein-ligand complex, which may help determine a mode of action of a ligand. Particular embodiments may be used to calculate a more accurate PMF score of a protein-ligand complex. In particular embodiments, a PMF score of a protein-ligand complex may be calculated according to more accurate PMF potentials that each account for multiple potentials (such as a van der Waals potential, an electrostatic potential, and a hydrogen bonding potential) that may cause repulsive force in a protein-ligand atom pair. Particular embodiments may be used to calculate a more accurate PMF potential between two atoms in a protein-ligand atom pair.

Certain embodiments may provide all, some, or none of these technical advantages. Certain embodiments may provide one or more other technical advantages, one or more of which may be readily apparent to those skilled in the art from the figures, descriptions, and claims herein.

### BRIEF DESCRIPTION OF THE DRAWINGS

To provide a more complete understanding of the present invention and the features and advantages thereof, reference is made to the following description, taken in conjunction with the accompanying drawings, in which:

FIG. 1 illustrates an example system for calculating a PMF score of a protein-ligand complex;

FIG. 2 illustrates an example table of empirically derived minimum binding-energy distance and well-depth values that may be used to calculated a PMF score of a protein-ligand complex; and

FIG. 3 illustrates an example method for calculating a PMF score of a protein-ligand complex.

### DETAILED DESCRIPTION OF THE INVENTION

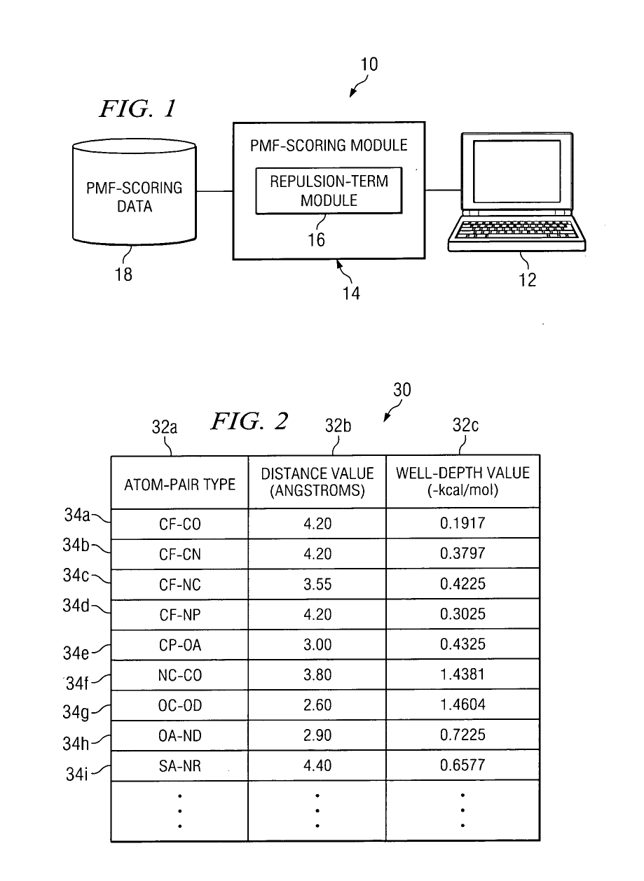

FIG. 1 illustrates an example system 10 for calculating a PMF score of a protein-ligand complex. System 10 includes a computer system 12 and a PMF-scoring module 14. In particular embodiments, a module may include software, hardware, or both. Computer system 12 may enable a user to provide input to and receive output from PMF-scoring module 14. Computer system 12 may include one or more modules for generating one or more graphical user interfaces (GUIs) for providing input to and receiving output from PMF-scoring module 14. PMF-scoring module 14 may calculate one or more PMF scores of one or more protein-ligand complexes specified by a user and return the calculated PMF scores to the user. A PMF score of a protein-ligand complex may indicate the binding affinity between the protein and the ligand in the protein-ligand complex, and the binding affinity between the protein and the ligand in the protein-ligand complex may indicate the ability of the ligand to inhibit or otherwise modify the function of the protein. PMF-scoring module 14 includes a repulsion-term module 16 that may calculate one or more repulsion terms, as described below. PMF-scoring module 14 may use PMF-scoring data 18 to calculate a PMF score of a protein-ligand complex. PMF-scoring data 18 data that PMF-scoring module 14 may use to calculate a PMF score of a protein-ligand complex. In particular embodiments, PMF-scoring data 18 includes empirically derived parameters (such as minimum binding-energy distance and well-depth values) that may be used to calculated a PMF score of a protein-ligand complex, as described below. Although components of system 10 are described and illustrated as being separate from each other, the present invention also contemplates any suitable components of system 10 being combined with any other suitable components in any suitable manner. As an example and not by way of limitation, in particular embodiments, PMF-scoring module 14 is executed at computer system 12. As another example, in particular embodiments, PMF-scoring data 18 is stored at computer system 12.

To calculate a PMF score of a protein-ligand complex, PMF-scoring module 14 calculates a PMF score of each protein-ligand atom pair in the protein-ligand complex and combines the calculated PMFs with each other. As an example and not by way of limitation, in particular embodiments:

$$PMF Score = {\sum }_{r < {r}_{cutoff}^{ij}}^{} kl {A}_{ij} ( r )$$

Aᵢⱼ(r) is a PMF of a protein-ligand atom pair of atom-pair type ij at distance r, and kl is a protein-ligand atom pair of atom-pair type ij. A protein-ligand atom pair of atom-pair type ij includes a first atom of protein atom type i and a second atom of ligand atom type j. A PMF of a protein-ligand atom pair corresponds to interaction energy between the two atoms in the protein-ligand atom pair. For purposes of calculating PMFs of protein-ligand atom pairs, protein atoms are defined by protein atom type and ligand atoms are defined by ligand atom type. Atom type is defined by element (carbon, oxygen, hydrogen, etc.) and local bonding environment (polar aliphatic, nonpolar aliphatic, polar aromatic, nonpolar aromatic, hydrogen bond donor, hydrogen bond acceptor, etc.). Examples of ligand atom types include nonpolar carbon sp³ aliphatic; polar sp³ carbon bonded to an atom other than carbon or hydrogen; sp nitrogen bound to one carbon; and other suitable ligand atom types. Examples of protein atom types include nonpolar aliphatic carbon; polar aliphatic sp² or sp³ carbon bonded to atoms other than carbon or hydrogen; positively charged nitrogen; sulfur as hydrogen bond acceptor; nitrogen in a planar ring structure; and other suitable protein atom types. In particular embodiments, there may be thirty-four ligand atom types and sixteen protein atom types. Herein, reference to atom type includes protein atom type, ligand atom type, or both, where appropriate.

PMFs of protein-ligand atom pairs are derived from application of one or more atom-pair distribution functions to data that describes analyzed protein-ligand complexes, such as data from the BROOKHAVEN PROTEIN DATA BANK (PDB) or the PDB maintained by the RESEARCH COLLABORATORY FOR STRUCTURAL BIOINFORMATCIS (RCSB). As an example and not by way of limitation, in particular embodiments:

$${A}_{ij} = - {k}_{B} T ln [ {f}_{vol\_corr}^{j} ( r ) \frac{{ρ}_{seg}^{ij} ( r )}{{ρ}_{bulk}^{ij}} ] = - {k}_{B} T ln {ρ}_{seg}^{ij} ( r ) - {k}_{B} T ln {f}_{vol\_corr}^{j} ( r ) + {k}_{B} T ln {ρ}_{bulk}^{ij}$$

k_{B} is a Boltzmann factor, T is absolute temperature,

$${f}_{vol\_corr}^{j} ( r )$$

is a ligand volume-correction factor, and ρ_{bulk}^{ij} is a number density of atom-pair type ij occurrences at a certain distance. In particular embodiments, to account for short-distance interaction between two atoms in a protein-ligand atom pair, a repulsion term is used to calculate a PMF of the protein-ligand atom pair. As an example and not by way of limitation, in particular embodiments, if two atoms in a protein-ligand atom pair of atom-pair type ij are separated from each other by a distance that is shorter than the longest distance without an occurrence of atom-pair type ij in data that describes analyzed protein-ligand complexes, a repulsion term is incorporated into the above formula. In particular embodiments, if short-distance interaction between two atoms in a protein-ligand atom pair is greater than 4 kcal/mol, the above formula is replaced by a repulsion term.

A repulsion term corresponds to repulsive force between two atoms in a protein-ligand atom pair. Repulsive force causes two atoms in a protein-ligand atom pair to repel each other and may result from van der Waals (VDW) potential, electrostatic potential, and hydrogen bond potential between the two atoms. Although repulsive force is described as resulting from particular potentials, the present invention contemplates repulsive force resulting from any suitable combination of any suitable potentials. In particular embodiments, a repulsion term used to calculate a PMF of a protein-ligand atom pair is calculated according to (1) a minimum binding-energy distance of the protein-ligand atom pair and (2) a well depth of the protein-ligand atom pair. A minimum binding-energy distance of a protein-ligand atom pair is a distance between the two atoms in the protein-ligand atom pair that corresponds to a minimum binding energy between the two atoms in the protein-ligand atom pair. A well depth of a protein-ligand atom pair corresponds to an amount of binding interaction between the two atoms in the protein-ligand atom pair.

In traditional PMF scoring, to calculate a repulsion term that may be used to calculate a PMF of a protein-ligand atom pair, a minimum binding-energy distance value is used that corresponds to a sum of VDW radii of the two atoms in the protein-ligand atom pair. In addition, in traditional PMF scoring, to calculate a repulsion term that may be used to calculate a PMF of a protein-ligand atom pair, a well-depth value is used that corresponds to atom hardnesses of the two atoms in the protein-ligand atom pair. VDW radii account for VDW potentials, but do not account for other potentials (such as electrostatic potential and hydrogen bond potential) that may cause repulsive force, as described above. As a result, traditional PMF scoring does not account for potentials other than VDW potential that may cause repulsive force and, therefore, often generates inaccurate PMF scores of protein-ligand complexes.

In contrast, in particular embodiments of the present invention, to calculate a repulsion term that may be used to calculate a PMF of a protein-ligand atom pair, repulsion-term module 16 may use a minimum binding-energy distance value that corresponds to an empirically derived minimum binding-energy distance value. In particular embodiments, to calculate a repulsion term that may be used to calculate a PMF of a protein-ligand atom pair, repulsion-term module 16 may use a well-depth value that corresponds to an empirically derived well-depth value. In particular embodiments, each atom-pair type may correspond to an empirically derived minimum binding-energy distance value and an empirically derived well-depth value. To calculate a repulsion term that may be used to calculate a PMF of a protein-ligand atom pair, repulsion-term module 16 may determine an atom-pair type of the protein-ligand atom pair, access an empirically derived minimum binding-energy distance value and an empirically derived well-depth value that correspond to the determined atom-pair type, and use the accessed values to calculate the PMF of the protein-ligand atom pair. As described above, empirically derived minimum binding-energy distance and well-depth values corresponding to atom-pair types may be stored in one or more tables of PMF scoring data 18.

FIG. 2 illustrates an example table 30 of empirically derived minimum binding-energy distance and well-depth values that may be used to calculated a PMF score of a protein-ligand complex. PMF scoring data 18 may include table 30. Column 32a corresponds to atom-pair type, column 32b corresponds to empirically derived minimum binding-energy distance, and column 32c corresponds to well-depth value. Rows 34 each correspond to an atom-pair type. The intersection of a row 34 and a column 32 defines a cell that may contain data. To determine an empirically derived minimum binding-energy distance value that may be used to calculate a repulsion term that may be used to calculate a PMF of a protein-ligand atom pair, PMF-scoring module 14 may access a column 34 corresponding to an atom-pair type of the protein-ligand atom pair and access an empirically derived minimum binding-energy distance value in row 32b and column 34. As an example and not by way of limitation, a protein-ligand atom pair has a first atom of protein atom type CF and a second atom of ligand atom type NP. PMF-scoring module 14 may access the cell at the intersection of column 34d and row 32b to determine an empirically derived minimum binding-energy distance value (4.2 angstroms) that may be used to calculate a repulsion term that may be used to calculate a PMF of the protein-ligand atom pair.

To determine an empirically derived well-depth value that may be used to calculate a repulsion term that may be used to calculate a PMF of a protein-ligand atom pair, PMF-scoring module 14 may access a column 34 corresponding to an atom-pair type of the protein-ligand atom pair and access an empirically derived minimum binding-energy distance value in row 32c and column 34. As an example and not by way of limitation, a protein-ligand atom pair has a first atom of protein atom type CF and a second atom of ligand atom type NC. PMF-scoring module 14 may access the cell at the intersection of column 34c and row 32c to determine an empirically derived well-depth value (0.4225 -kcal/mol) that may be used to calculate a repulsion term that may be used to calculate a PMF of the protein-ligand atom pair. Although a particular table of particular empirically derived minimum binding-energy distance and well-depth values is described and illustrated, the present invention contemplates any suitable table (or other suitable data structure) of any suitable empirically derived minimum binding-energy distance and well-depth values.

In particular embodiments, to generate empirically derived minimum binding-energy distance and well-depth values that may be used to calculate repulsion terms, multiple sets of minimum binding-energy distance and well-depth values corresponding to multiple atom-pair types are generated, used to calculate PMF scores of multiple analyzed protein-ligand complexes, and compared with actual, measured binding affinities of the analyzed protein-ligand complexes. Minimum binding-energy distance and well-depth values in a generated set of minimum binding-energy distance and well-depth values that yields a best agreement with the actual measured binding affinities of the analyzed protein-ligand complexes may be used by repulsion-term module 16 to calculate repulsion terms that may be used to calculate PMFs of protein-ligand atom pairs.

As an example and not by way of limitation, in particular embodiments, a first set of minimum binding-energy distance and well-depth values is generated. Each atom-pair type has a minimum binding-energy distance value and a well-depth value in a generated set of minimum binding-energy distance and well-depth values. One or more of the values in the first set may be generated manually. One or more of the values in the first set may be generated automatically. One or more of the values in the first set may be generated according to a random process (such as a genetic algorithm). A genetic algorithm may be executed automatically by a computer system. The first set of minimum binding-energy distance and well-depth values is then used to calculate a PMF score of each of multiple protein-ligand complexes. The calculated PMF scores are then used to predict the structure of each of the protein-ligand complexes.

The predicted structures of the protein-ligand complexes are compared with actual, analyzed structures of the protein-ligand complexes. In particular embodiments, a root mean square (RMS) deviation between the predicted structure and the actual, analyzed structure of each of the protein-ligand complexes is calculated. An RMS deviation between a predicted structure and an actual, analyzed structure of a protein-ligand complex indicates one or more differences in atom position between the predicted structure and the actual, analyzed structure of the protein-ligand complex. If there is no difference in atom position between the predicted structure and the actual, analyzed structure, the RMS deviation between the two structures is zero. An average of the RMS deviations of the first set of values may be calculated and used to gauge agreement between the first set of values and the actual repulsive forces in the atom-pair types.

A second set of minimum binding-energy distance and well-depth values is then generated, and an average RMS deviation of the second set of values is calculated, as described above. The average RMS deviation of the first set of values is then compared with the average RMS deviation of the second set of values. If the average RMS deviation of the first set of values is less than the average RMS deviation of the second set of values, the first set of values may include one or more minimum binding-energy distance values, well-depth values, or both that are preferable to one or more minimum binding-energy distance values, well-depth values, or both in the second set of values. If the average RMS deviation of the first set of values is greater than the average RMS deviation of the second set of values, the second set of values may include one or more minimum binding-energy distance values, well-depth values, or both that are preferable to one or more minimum binding-energy distance values, well-depth values, or both in the first set of values. If the average RMS deviations of the first and second sets of values are equal to each other, the first set of values may include one or more minimum binding-energy distance values, well-depth values, or both that are equivalent to one or more minimum binding-energy distance values, well-depth values, or both in the second set of values.

One or more of the values in the second set of values may be generated manually. As an example and not by way of limitation, a user may review the results of the first set of values and modify one or more minimum binding-energy distance values, well-depth values, or both in the first set of values to generate the second set of values. One or more of the values in the second set of values may be generated automatically. One or more of the values in the second set of values may be generated according to a random process (such as a genetic algorithm). As an example and not by way of limitation, a genetic algorithm may be applied to the first set of values to generate the second set of values.

One or more additional sets of minimum binding-energy distances and well-depth values may be generated, and an average RMS deviation of each additional set of values may be calculated, as described above. The average RMS deviations may be compared with each other to identify one or more sets of minimum binding-energy distance and well-depth values that yields a best agreement with actual measured structures, binding affinities, or both of analyzed protein-ligand complexes. One or more of the values in an additional set of values may be generated manually. As an example and not by way of limitation, a user may review the results of one or more preceding sets of values and modify one or more minimum binding-energy distance values, well-depth values, or both in one or more of the one or more preceding sets of values to generate the additional set of values. One or more of the values in an additional set of values may be generated automatically. One or more of the values in an additional set of values may be generated according to a random process (such as a genetic algorithm). As an example and not by way of limitation, a genetic algorithm may be applied to one or more preceding sets of values to generate the additional set of values. In particular embodiments, over multiple generations of value sets, a set of values may eventually be generated that yields a best (or even best possible) agreement with actual measured binding affinities of the analyzed protein-ligand complexes.

Any suitable number of value sets may be generated. As an example and not by way of limitation, a predetermined number of sets of minimum binding-energy and well-depth values may be generated. As another example, sets of minimum binding-energy and well-depth values may be generated until an average RMS deviation below predetermined threshold is reached. As another example, sets of minimum binding-energy and well-depth values may be generated until a predetermined rate of decrease in average RMS deviation is reached. The predetermined rate of decrease in average RMS deviations may correspond to a point at which generating further value sets is unlikely to yield substantially better agreement with actual measured binding affinities of analyzed protein-ligand complexes. In particular embodiments, minimum binding-energy and well-depth values (and possibly other parameters) may be determined according to deviations between predicted trends and measured trends in data describing binding affinity and activity in analyzed protein-ligand complexes.

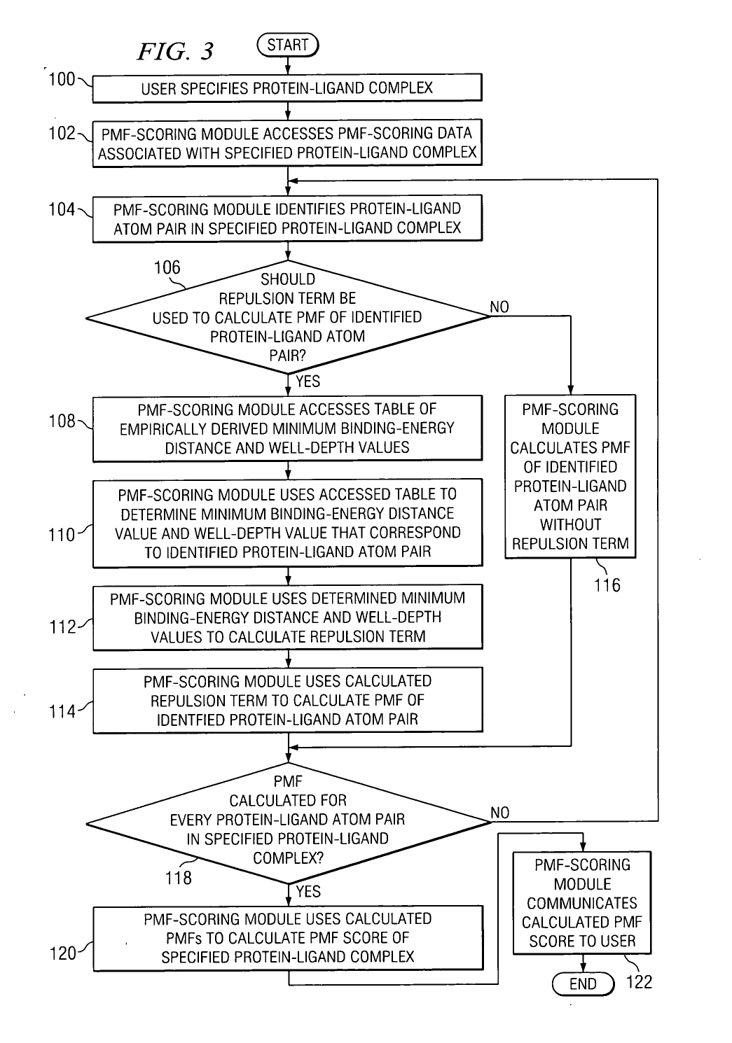

FIG. 3 illustrates an example method for calculating a PMF score of a protein-ligand complex. The method begins at step 100, where a user at computer system 12 specifies a protein-ligand complex for PMF scoring. At step 102, PMF-scoring module 14 accesses PMF-scoring data 18 associated with the specified protein-ligand complex. PMF-scoring data 18 may describe the protein and the ligand in the specified protein-ligand complex. As an example, PMF-scoring data 18 may describe the number of atoms and the type and position of each atom in the protein. As another example, PMF-scoring data 18 may describe the number of atoms and the type and position of each atom in the ligand. At step 104, PMF-scoring module 14 identifies a protein-ligand atom pair in the specified protein-ligand complex. At step 106, if a repulsion term should be used to calculate a PMF of the identified protein-ligand atom pair, the method proceeds to step 108.

At step 108, PMF-scoring module 14 accesses a table 30 of empirically derived minimum binding-energy distance and well-depth values. PMF-scoring data 18 may include table 30. At step 110, PMF-scoring module 14 uses table 30 to determine a minimum binding-energy distance value and a well-depth value that correspond to the identified protein-ligand atom pair. At step 112, PMF-scoring module 14 uses the determined minimum binding-energy distance and well-depth values to calculate a repulsion term. At step 114, PMF-scoring module 14 uses the calculated repulsion term to calculate a PMF of the identified protein-ligand atom pair, at which point the method proceeds to step 118. At step 106, if a repulsion term should not be used to calculate a PMF of the identified protein-ligand atom pair, the method proceeds to step 116.

At step 116, PMF-scoring module 14 calculates a PMF of the identified protein-ligand atom pair without a repulsion term. At step 118, if a PMF of a protein-ligand atom pair in the specified protein-ligand complex has not been calculated, the method returns to step 104. At step 118, if a PMF of each protein-ligand atom pair in the specified protein-ligand complex has been calculated, the method proceeds to step 120. At step 120, PMF-scoring module 14 uses the calculated PMFs of the protein-ligand atom pairs in the specified protein-ligand complex to calculate a PMF score of the specified protein-ligand complex. At step 122, PMF-scoring module 14 communicates the calculated PMF score to the user at computer system 12, at which point the method ends. Although particular steps of the method illustrated in FIG. 3 are described and illustrated as occurring in a particular order, the present invention contemplates any suitable steps of the method described above occurring in any suitable order.

Although the present invention has been described with several embodiments, myriad changes, variations, alterations, transformations, and modifications may be suggested to one skilled in the art, and it is intended that the present invention encompass such changes, variations, alterations, transformations, and modifications as fall within the scope of the appended claims. The present invention is not intended to be limited, in any way, by any statement in the specification that is not reflected in the claims.

1. A system for calculating a potential of mean force (PMF) score of a protein-ligand complex, the system comprising:
a repulsion-term module that:
accesses one or more parameters useable to calculate a repulsion term useable to calculate a PMF of a protein-ligand atom pair in the protein-ligand complex, the one or more parameters corresponding to an atom-pair type of the protein-ligand atom pair;
using the one or more accessed parameters, calculates the repulsion term useable to calculate the PMF of the protein-ligand atom pair; and
communicates the calculated repulsion term for calculation of the PMF score of the protein-ligand complex.

2. The system of claim 1, wherein one of the parameters corresponding to the atom-pair type of the protein-ligand atom pair comprises an empirically derived minimum binding-energy distance value corresponding to the atom-pair type of the protein-ligand atom pair.

3. The system of claim 1, wherein one of the parameters corresponding to the atom-pair type of the protein-ligand atom pair comprises an empirically derived well-depth value corresponding to the atom-pair type of the protein-ligand atom pair.

4. The system of claim 1, wherein:
a first one of the parameters corresponding to the atom-pair type of the protein-ligand atom pair comprises an empirically derived minimum binding-energy distance value corresponding to the atom-pair type of the protein-ligand atom pair; and
a second one of the parameters corresponding to the atom-pair type of the protein-ligand atom pair comprises an empirically derived well-depth value corresponding to the atom-pair type of the protein-ligand atom pair.

5. The system of claim 4, wherein a set of a plurality of empirically derived minimum binding-energy distance and well-depth values corresponding to a plurality of protein-ligand atom pairs comprises the empirically derived minimum binding-energy distance and well-depth values corresponding to the atom-pair type of the protein-ligand atom pair, the set of empirically derived minimum binding-energy distance and well-depth values yielding a best agreement with a plurality of actual analyzed protein-ligand atom pairs.

6. The system of claim 5, wherein the plurality of actual analyzed protein-ligand atom pairs are described in a protein data bank (PDB).

7. The system of claim 5, wherein the best agreement between the set of empirically derived minimum binding-energy distance and well-depth values and the plurality of analyzed protein-ligand atom pairs is determined according to a plurality of root mean square (RMS) deviations between:
protein-ligand complex structures predicted according to the set of empirically derived minimum binding-energy distance and well-depth values; and
actual analyzed protein-ligand complex structures corresponding to the predicted protein-ligand complex structures;

8. The system of claim 7, wherein a plurality of sets of empirically derived minimum binding-energy distance and well-depth values are generated and compared with each other to determine the set of empirically derived minimum binding-energy distance and well-depth values yielding the best agreement with the plurality of actual analyzed protein-ligand atom pairs.

9. The system of claim 7, wherein one or more of the plurality of sets of empirically derived minimum binding-energy distance and well-depth values are generated according to one or more of:
one or more manual processes; and
one or more automatic processes.

10. The system of claim 9, wherein one of the automatic processes comprises execution of a genetic algorithm.

11. A method for calculating a potential of mean force (PMF) score of a protein-ligand complex, the method comprising:
accessing one or more parameters useable to calculate a repulsion term useable to calculate a PMF of a protein-ligand atom pair in the protein-ligand complex, the one or more parameters corresponding to an atom-pair type of the protein-ligand atom pair;
using the one or more accessed parameters, calculating the repulsion term useable to calculate the PMF of the protein-ligand atom pair; and
communicating the calculated repulsion term for calculation of the PMF score of the protein-ligand complex.

12. The method of claim 11, wherein one of the parameters corresponding to the atom-pair type of the protein-ligand atom pair comprises an empirically derived minimum binding-energy distance value corresponding to the atom-pair type of the protein-ligand atom pair.

13. The method of claim 11, wherein one of the parameters corresponding to the atom-pair type of the protein-ligand atom pair comprises an empirically derived well-depth value corresponding to the atom-pair type of the protein-ligand atom pair.

14. The method of claim 11, wherein:
a first one of the parameters corresponding to the atom-pair type of the protein-ligand atom pair comprises an empirically derived minimum binding-energy distance value corresponding to the atom-pair type of the protein-ligand atom pair; and
a second one of the parameters corresponding to the atom-pair type of the protein-ligand atom pair comprises an empirically derived well-depth value corresponding to the atom-pair type of the protein-ligand atom pair.

15. The method of claim 14, wherein a set of a plurality of empirically derived minimum binding-energy distance and well-depth values corresponding to a plurality of protein-ligand atom pairs comprises the empirically derived minimum binding-energy distance and well-depth values corresponding to the atom-pair type of the protein-ligand atom pair, the set of empirically derived minimum binding-energy distance and well-depth values yielding a best agreement with a plurality of actual analyzed protein-ligand atom pairs.

16. The method of claim 15, wherein the plurality of actual analyzed protein-ligand atom pairs are described in a protein data bank (PDB).

17. The method of claim 15, wherein the best agreement between the set of empirically derived minimum binding-energy distance and well-depth values and the plurality of analyzed protein-ligand atom pairs is determined according to a plurality of root mean square (RMS) deviations between:
protein-ligand complex structures predicted according to the set of empirically derived minimum binding-energy distance and well-depth values; and
actual analyzed protein-ligand complex structures corresponding to the predicted protein-ligand complex structures.

18. The method of claim 17, wherein a plurality of sets of empirically derived minimum binding-energy distance and well-depth values are generated and compared with each other to determine the set of empirically derived minimum binding-energy distance and well-depth values yielding the best agreement with the plurality of actual analyzed protein-ligand atom pairs.

19. The method of claim 17, wherein one or more of the plurality of sets of empirically derived minimum binding-energy distance and well-depth values are generated according to one or more of:
one or more manual processes; and
one or more automatic processes.

20. The method of claim 19, wherein one of the automatic processes comprises execution of a genetic algorithm.

21. Software for calculating a potential of mean force (PMF) score of a protein-ligand complex, the software embodied in computer-readable media and when executed operable to:
access one or more parameters useable to calculate a repulsion term useable to calculate a PMF of a protein-ligand atom pair in the protein-ligand complex, the one or more parameters corresponding to an atom-pair type of the protein-ligand atom pair;
using the one or more accessed parameters, calculate the repulsion term useable to calculate the PMF of the protein-ligand atom pair; and
communicate the calculated repulsion term for calculation of the PMF score of the protein-ligand complex.

22. The software of claim 21, wherein one of the parameters corresponding to the atom-pair type of the protein-ligand atom pair comprises an empirically derived minimum binding-energy distance value corresponding to the atom-pair type of the protein-ligand atom pair.

23. The software of claim 21, wherein one of the parameters corresponding to the atom-pair type of the protein-ligand atom pair comprises an empirically derived well-depth value corresponding to the atom-pair type of the protein-ligand atom pair.

24. The software of claim 21, wherein:
a first one of the parameters corresponding to the atom-pair type of the protein-ligand atom pair comprises an empirically derived minimum binding-energy distance value corresponding to the atom-pair type of the protein-ligand atom pair; and
a second one of the parameters corresponding to the atom-pair type of the protein-ligand atom pair comprises an empirically derived well-depth value corresponding to the atom-pair type of the protein-ligand atom pair.

25. The software of claim 24, wherein a set of a plurality of empirically derived minimum binding-energy distance and well-depth values corresponding to a plurality of protein-ligand atom pairs comprises the empirically derived minimum binding-energy distance and well-depth values corresponding to the atom-pair type of the protein-ligand atom pair, the set of empirically derived minimum binding-energy distance and well-depth values yielding a best agreement with a plurality of actual analyzed protein-ligand atom pairs.

26. The software of claim 25, wherein the plurality of actual analyzed protein-ligand atom pairs are described in a protein data bank (PDB).

27. The software of claim 25, wherein the best agreement between the set of empirically derived minimum binding-energy distance and well-depth values and the plurality of analyzed protein-ligand atom pairs is determined according to a plurality of root mean square (RMS) deviations between:
protein-ligand complex structures predicted according to the set of empirically derived minimum binding-energy distance and well-depth values; and
actual analyzed protein-ligand complex structures corresponding to the predicted protein-ligand complex structures.

28. The software of claim 27, wherein a plurality of sets of empirically derived minimum binding-energy distance and well-depth values are generated and compared with each other to determine the set of empirically derived minimum binding-energy distance and well-depth values yielding the best agreement with the plurality of actual analyzed protein-ligand atom pairs.

29. The software of claim 27, wherein one or more of the plurality of sets of empirically derived minimum binding-energy distance and well-depth values are generated according to one or more of:
one or more manual processes; and
one or more automatic processes.

30. The software of claim 29, wherein one of the automatic processes comprises execution of a genetic algorithm.

31. A system for calculating a potential of mean force (PMF) score of a protein-ligand complex, the system comprising:
means for accessing one or more parameters useable to calculate a repulsion term useable to calculate a PMF of a protein-ligand atom pair in the protein-ligand complex, the one or more parameters corresponding to an atom-pair type of the protein-ligand atom pair;
means for, using the one or more accessed parameters, calculating the repulsion term useable to calculate the PMF of the protein-ligand atom pair; and
means for communicating the calculated repulsion term for calculation of the PMF score of the protein-ligand complex.
